(19) United States
(12) Patent Application Publication
(10) Pub. No.: US 2006/0205936 A1
(43) Pub. Date: Sep. 14, 2006

(54) Chlorination of Sucrose-6-esters

(75) Inventors: Lizhu Jia, Bedminster, NJ (US); Song Zhu, Bedminster, NJ (US)

Correspondence Address: SL LABORATORIES, LLC, 1430 ROUTE 206, SUITE 210, BEDMINSTER, NJ, 07921, US

(73) Assignee: SL LABORATORIES, LLC, Bedminster, NJ (US)

(21) Appl. No.: 10/906,951

(22) Filed: Mar. 14, 2005

(51) Int. Cl. C13K 5/00; C07H 3/04

(52) U.S. Cl. 536/123.13

(57) ABSTRACT

An improved process for chlorination of sugars produces chlorodeoxy derivatives, in particular the chlorination of sucrose-6-esters to produce sucralose (4,1′,6′-trichloro-4,1′,6′-trideoxy galactosucrose), with thionyl chloride and N,N-dimethyl formamide (DMF) at the ration of about 1 molar equivalent of thionyl chloride for every molar equivalent of free hydroxyl group.

This invention relates to the improved process for chlorination of sugars to produce chlorodeoxy derivatives, in particular to the chlorination of sucrose-6-esters to produce sucralose (4,1′, 6′-trichloro-4,1′,6′-trideoxy galactosucrose).

### BACKGROUD OF THE INVENTION

There are many processes for the preparation of sucralose. Chlorination of sucrose-6-esters is one of the most widely used methods.

Chlorination of alcohols using thionyl chloride and organic base, such as pyridine, has been known for a long time. Thionyl chloride can rapidly react with two alcohol molecules to form a sulphite. The HCl generated from the reaction can be neutralized by organic bases. Further reaction of thionyl chloride with sulphite produces chlorosulphite (Gerrard, J. Chem. Soc. 1939, 998; 1940, 218; 1944, 85). Chlorosulphite react with HCl salts provide chlorination product. The thionyl chloride/pyridine chlorination agent was initially used in the preparation of sucralose. However, this process produced many unwanted by-products due to the possible chlorination of organic amines, which would give low yield for desired product and increase the difficulties for purification. The triphenylphosphine oxide/thionyl chloride reagent can be used in an inner solvent such as toluene to give an improved yield, but this process needs excess triphenylphosphine oxide for the chlorination, which would generate too much waste and increase difficulties to remove triphenylphosphine oxide from the chlorinated product. The modified process by using triarylphosphine oxide or sulphide do not give many advantages due to economic reasons and the difficulties for the purification. Vilsmeier-type reagents provide an alternate method for the chlorination of sucrose-6-esters. There are two types of chemicals were used to make Vilsmeier-type reagents in the chlorination of sucrose-6-esters (U.S. Pat. No. 4,980,463 and WO 99/60006). Type I are phosphorus reagents, including phosphorus pentachloride and phosphorus oxichloride. Vilsmeier reagents generated from this type of chemicals needed extensive wash to remove phosphorus related by-product, which resulted low yield for desired Vilsmeier reagents. Producing dark colored reaction mixtures and intractable by-products are further disadvantages for the chlorination by using this type of Vilsmeier reagents. Type II chemicals are acid chlorides. Widely used acid chlorides in the chlorination of sucrose-6-esters are phosgene, thionyl chloride and oxalyl chloride. The processes for all existing chlorination of sucrose-6-esters by using this type of Vilsmeier reagents are followed the procedures described below:
1. Dissolve sucrose-6-ester in N,N-dimethylformamide (DMF) at 10 to 100° C.
2. Cool the reaction mixture to −50 to −10° C., and treated drop wise over a period time with acid chloride. A thick precipitate would be formed. Presumably the thick precipitate was Vilsmeier reagent.
3. The reaction mixture was heated to certain temperature stepwise. Chlorination of sucrose-6-esters was done after certain amount of time at temperature of 100 to 130° C.

Utilization above procedure for type II Vilsmeier reagent in the chlorination of sucrose-6-esters, phosgene is too toxic, oxalyl chloride is economically constrained, and both phosgene and oxalyl chloride generate pollution concerns and operation difficulties due to violence reaction with DMF. As for thionyl chloride, due to lack activity with DMF at low temperature, the compound tend to react with hydroxy groups in sucrose-6-ester to form a sulphite, as described above. This sulphite is precipitate at low temperature, and can be dissolved in DMF when temperature reaches certain degree. Meanwhile, a series complex mixture of products is produced due to the competition reaction for thionyl chloride with alcohols and DMF, which resulting lower yield for desired chlorinated product.

### SUMMARY OF THE INVENTION

We have now found an improve process for the chlorination of sucrose-6-esters by Vilsmeier-type reagent, generated from economically favored thionyl chloride to obtain excellent yields of the desired chlorodeoxy sugar derivatives with easy to handle process.

There are 7 free hydroxy groups in sucrose-6-esters; each of hydroxy group should have interaction, or reaction with 1 Vilsmeier reagent. That is why at least 7 molar equivalents of Vilsmeier reagent is needed for the chlorination of sucrose-6-ester, which translated that 7 molar equivalents of thionyl chloride is needed for the chlorination.

DMF is not only the solvent for the reaction, but also a reactant to react with thionyl chloride to form Vilsmeier reagent. In practice, the amount of DMF can vary to a certain extent, favorably by using 2-6 mL for every 1 mL thionyl chloride. The most favorably ratio is 3-4:1 by volume.

At the low temperature, such as 0 to −50° C., favorably 0 to −10° C., thionyl chloride was added to DMF (ratio 4:1) slowly. No precipitate would be observed. The reaction mixture was heated to certain temperature for certain time for the formation of Vilsmeier reagent. The reaction mixture was then cooled to bellow 50° C., favorably 10-40° C., and vacuum distillation to remove sulphur dioxide, which was generated from the reaction.

The reaction temperature to form a Vilsmeier reagent is crucial for completing the reaction. The temperature should be ranged from 0 to 100° C., favorably 20 to 80° C.; the most favorable temperature for the reaction is 30-60° C. Higher temperature can destroy desired Vilsmeier reagent, but lower temperature slow down the reaction.

Applying vacuum to remove sulphur dioxide is also important for clearer reaction. After removing sulphur dioxide, white precipitate of Vilsmeier reagent can be observed. The Vilsmeier reagent can be used directly with sucrose-6-esters, or filtered and dried to give high quality Vilsmeier reagent and stored for later usage.

According to the present invention, we provide an improved process for the chlorination sucrose-6-esters by using economically favored thionyl chloride and DMF, comprising about one molar equivalent of thionyl chloride for every molar equivalent of free hydroxyl in the sugars. The method of the present invention provides an efficient, practical, high selective and clean reaction with easy to control in production comparing with other existing inventions for the chlorination of sugars.

### EXAMPLE 1

1.5 Gram Scale Chlorination of Sucrose-6-Acetate with Vilsmeier Reagent Generated by Thionyl Chloride and DMF In Situ without Filtration

Dimethylformamide (DMF, 8 mL) was added thionyl chloride (2 mL, 27 mmol) at −10° C. with stirring. Heat was released, but no precipitate was observed. The reaction mixture was stirred at 50° C. for 2 hr. Vacuum distillation removed 1 mL DMF at the temperature 40-60° C. The reaction mixture was then cooled to 0° C. while white solid Vilsmeier reagent was crystallized. Sucrose 6-acetate (1.5 g, 85% purity, 3.3 mmol) was dissolved in 5 mL DMF and added to above Vilsmeier reagent at 0° C. After completing addition, the reaction mixture was stirred for 30 min. at 0 to 10° C. The reaction mixture was then heated to 60° C. and stirred at this temperature for 1 hr. The reaction mixture was heated to 110-115° C. and hold at this temperature for 2 hr. The reaction mixture was cooled to 0° C. and added 5 mL ice cold 4 N NaOH. The pH value for the reaction mixture was adjusted to 7 by adding certain amount of 2 N hydrochloric acid or NaOH. 20 mL EtOAc was added. The organic layer was separated. The aqueous layer was extracted with EtOAc (2×15 mL). The combined organic layers were washed with water. The solvent was removed. The residue was high vacuum dried to give 1.3 g crude sucralose 6-acetate with 68% purity.

### EXAMPLE 2

30 Gram Scale Chlorination of Sucrose-6-Acetate with Vilsmeier Reagent Generated by Thionyl Chloride and DMF In Situ without Filtration

Dimethylformamide (DMF, 160 mL) was added thionyl chloride (40 mL, 543 mmol) at −10° C. with stirring. Heat was released, but no precipitate was observed. The reaction mixture was stirred at 50° C. for 2 hr. Vacuum distillation removed 10 mL DMF at the temperature 40-60° C. The reaction mixture was then cooled to 0° C. while white solid Vilsmeier reagent was crystallized. Sucrose 6-acetate (30 g, 85% purity, 66 mmol) was dissolved in 100 mL DMF and added to above Vilsmeier reagent at 0° C. After completing addition, the reaction mixture was stirred for 30 min. at 0 to 10° C. The reaction mixture was then heated to 60° C. and stirred at this temperature for 1 hr. The reaction mixture was heated to 110-115° C. and hold at this temperature for 3 hr. The reaction mixture was cooled to 0° C. and added 100 mL ice cold 4 N NaOH. The pH value for the reaction mixture was adjusted to 7 by adding certain amount of 2 N hydrochloric acid or NaOH. 500 mL EtOAc was added. The organic layer was separated. The aqueous layer was extracted with EtOAc (2×300 mL). The combined organic layers were washed with water. The solvent was removed. The residue was high vacuum dried to give 26 g crude Sucralose 6-acetate with 72% purity.

### EXAMPLE 3

Chlorination of Sucrose-6-Acetate with Solid Vilsmeier Reagent Generated by Thionyl Chloride and DMF After Filtration

Dimethylformamide (DMF, 80 mL) was added thionyl chloride (50 mL, 679 mmol) at −10° C. with stirring. The reaction mixture was stirred at 50° C. for 2 hr. Vacuum distillation removed 2 mL DMF at the temperature 40-60° C. or until a colourless hygroscopic solid was observed. The reaction mixture was added 300 mL dichloromethane, then cooled to 0° C. and quickly filtered with suction. The white filter cake was rinsed with dichloromethane (2×100 mL), and dried to afford 72 g white solid Vilsmeier reagent. Sucrose 6-acetate (30 g, 85% purity, 66 mmol) was dissolved in 100 mL DMF. To this reaction mixture, the above Vilsmeier reagent was added portion wise at 0° C. After completing addition, the reaction mixture was stirred for 30 min. at this temperature. The reaction mixture was heated to 60° C. and stirred for 1 hr. The reaction mixture was then heated to 110-115° C. and hold at this temperature for 3 hr. The reaction mixture was cooled to 0° C. and added 100 mL ice cold 4 N NaOH. The pH value for the reaction mixture was adjusted to 7. 20 mL EtOAc was added. The organic layer was separated. The aqueous layer was extracted with EtOAc (2×300 mL). The combined organic layers were washed with water. The solvent was removed. The residue was high vacuum dried to give 27 g crude Sucralose 6-acetate with 73% purity.

What is claimed is:

1. An improved process for the chlorination of sucrose and partly protected sucrose derivatives, such as sucrose-6-esters, to produce sucralose (4,1′,6′-trichloro-4,1′,6′-trideoxy galactosucrose) which comprises the steps as follows:
(a). Thionyl chloride was slowly added to tertiary amide at certain temperature, preferable at −30 to 80° C., more preferable at −10 to 50° C., and the most preferable at −10 to 10° C.;
(b). The reaction mixture in step (a) was stirred at temperature of 0 to 100° C. for 1-6 hours;
more preferable at 20 to 80° C. for 1-6 hours, and the most preferable at 30 to 50° C. for 1-6 hours;
(c). The reaction mixture in step (b) was evaporated in vacuo for 1-4 hours at temperature bellow 100° C., preferable bellow 80° C., and the most preferable at temperature bellow 60° C., until all of sulphur dioxide was removed. In practical, slowly removing 2-10% of DMF by vacuum distillation at temperature at 30 to 50° C. would be good for this process;
(d). The sulphur dioxide that generated from the reaction can also be removed by other means, such as bubbling gas, or gases, in the reaction mixture;
(e). The reaction mixture in step (c) was cooled to certain temperature, preferable at −30 to 80° C., more preferable at −10 to 50° C., and the most preferable at −10 to 10° C. The solid Vilsmeier-type reagent can be used directly (one pot reaction), or filtered and dried for stepwise reaction for the chlorination;
(f). A solution of sucrose-6-esters was slowly added to above reaction mixture at the temperature bellow 100° C., preferable at −30 to 80° C., more preferable at −10 to 50° C., and the most preferable at −10 to 10° C.;
(g). The temperature of the reaction mixture was increased to not higher than 100° C. for 0.1-6 hours; preferable at 20 to 80° C. for 1-6 hours, and more preferable at 50 to 80° C. for 1-6 hours;
(h). The temperature of the reaction mixture was further increased to not higher than 140° C. for 0.1-6 hours; preferable at 80 to 130° C. for 1-6 hours, and more preferable at 100 to 120° C. for 1-6 hours, or hold a period time sufficient to produce sucralose-6-ester (4,1′,6′-trichloro-4,1′,6′-trideoxy galactosucrose-6-ester).

2. The process of claim 1 wherein said tertiary amide contain a N-formyl group.

3. The process of claim 2 wherein said tertiary amide is N,N-dimethylformamide (DMF).

4. The process of claim 1 wherein said evaporated in vacuo for 1-4 hours is present in removing sulphur dioxide, which generated from the reaction.

5. The process of claim 1 wherein said gas, or gases, means nitrogen. argon, carbon dioxide, dry air, or other suitable gas, or a mixture of gases.

6. The process of claim 1, in which the thionyl chloride used in the stage, is present in molar excess.

7. The process of claim 1, in which thionyl chloride is present in an amount of about 0.9 to 1.75 molar equivalent per free hydroxy group.

8. The process of claim 1 wherein said partly protected sucrose derivative is 6-protected sucrose.

9. The process of the claim 8 wherein said 6-protected sucrose is selected from the group consisting 6-esters, 6-ether and a 6,4-diester.

10. The process of claim 9 wherein said 6-protected sucrose is selected from a group of 6-acetate, 6-benzoate and raffinose.

11. The process for the preparation of sucralose, comprising chlorination of sucrose-6-esters by using Vilsmeier-type reagent by a process according claim 1 to form a sucralose-6-ester, esterification and de-esterification of the pentaester to form sucralose.
